(19) United States
(12) Patent Application Publication
(10) Pub. No.: US 2011/0184243 A1
(43) Pub. Date: Jul. 28, 2011

(54) ENDOSCOPE WITH DIFFERENT COLOR LIGHT SOURCES

(75) Inventors: George Wright, Dove Canyon, CA (US); Lonnie Hoyle, Mission Viejo, CA (US); Kais Almarzouck, Tustin, CA (US)

(73) Assignee: INTEGRATED ENDOSCOPY, INC., Rancho Santa Margarita, CA (US)

(21) Appl. No.: 12/975,060

(22) Filed: Dec. 21, 2010

Related U.S. Application Data
(60) Provisional application No. 61/289,240, filed on Dec. 22, 2009.

(51) Int. Cl. A61B 1/06

(52) U.S. Cl. 600/180

(57) ABSTRACT

Disclosed are systems and methods involving an endoscope system having a plurality of different illumination sources. Such illumination sources can be controlled to provide various illumination effects. In certain embodiments, illumination sources can be controlled so that resulting outputs can be combined to yield, for example, an intensity profile that approximates a desired source such as a Xenon lamp. In certain embodiments, illumination sources can be activated in sequence so as to allow, for example, use of a monochromatic detector to generate a color image. In certain embodiments, illumination sources can be controlled so as to allow switching between two or more illumination modes.

### CROSS-REFERENCE TO RELATED APPLICATIONS

The present application claims priority from U.S. patent application Ser. No. 61/289,240, filed Dec. 22, 2009, the content of which is incorporated herein by reference in its entirety.

### BACKGROUND

1. Field of the Invention

The present disclosure relates generally to medical devices and methods, and more particularly, to endoscopes and similar devices having different color light sources.

2. Description of the Related Art

Endoscopes typically include a tube dimensioned to be insertable into a body. Once inserted to a region of interest, light is provided to illuminate an object to be viewed. The illuminated object is then detected and imaged by a detector.

### SUMMARY

In certain embodiments, the present disclosure relates to an endoscope system having a probe configured to be insertable into a body. The system further includes a plurality of different illumination sources configured and disposed relative to the probe so as to provide illumination having one or more desired properties from the probe to an object inside the body. The system further includes an assembly of optical elements configured and disposed relative to the probe so as to form images of the illuminated object. The system further includes a detector configured to detect the images and generate signals representative of the detected images. The system further includes a controller configured to control operation of the illumination sources so as to yield the one or more desired properties of the illumination.

In certain embodiments, the present disclosure relates to a method for configuring an endoscope. The method includes providing a plurality of different illumination sources. The method further includes providing a control mechanism configured to control each of the illumination sources to achieve a desired illumination configuration.

In certain embodiments, the present disclosure relates to a method for operating an endoscope. The method includes providing a plurality of different color light sources. The method further includes activating the different color light sources in a manner that yields a selected configuration of illumination provided to an object being examined. The method further includes obtaining an image of the object during at least a portion of the illumination.

### BRIEF DESCRIPTION OF THE DRAWINGS

FIG. 1 shows a block diagram of an endoscope system having various components configured to facilitate one or more features of the present disclosure;

FIG. 2 shows that in certain embodiments, an endoscope can be coupled electrically and/or optically to a separate component via a cable so as to facilitate transfer of, for example, power and/or signals associated with images detected by the endoscope;

FIG. 3 shows that in certain embodiments, an endoscope can be coupled to a separate component without a cable so as to facilitate transfer of, for example, control signals and/or signals associated with images detected by the endoscope;

FIG. 4 shows that in certain embodiments, the endoscope system of FIG. 1 can include a plurality of different color light sources disposed on a portion of the endoscope;

FIGS. 5A and 5B show non-limiting examples of how the different colored light sources can be arranged;

FIG. 6 shows a block diagram of the endoscope system of FIG. 1 in operation, where a plurality of different color light sources can provide different illumination configurations to an object being observed;

FIG. 7A shows that in certain embodiments, two or more different color light sources of FIG. 6 can be controlled separately;

FIG. 7B shows an example configuration of the illumination control of FIG. 6, where the two or more different color light sources can include red (R), green (G), and blue (B) light-emitting diodes (LEDs);

FIG. 8 schematically illustrates that in certain embodiments, one or more operating parameters of one or more of the plurality of color light sources can be adjusted such that lights from the color light sources can combine to yield or approximate a desired intensity distribution;

FIG. 9 shows that in certain embodiments, a plurality of illumination sources can be provided and controlled as described herein, and such sources can include emission wavelengths in ultraviolet, visible, and/or infrared ranges;

FIG. 10 shows that in certain embodiments, two or more illumination sources in the visible range can be provided and controlled so as to yield a desired standardized chromaticity;

FIG. 11 shows an example process that can be implemented to allow control of the plurality of illumination sources;

FIG. 12 shows that in certain embodiments, a process can be implemented such that two or more of the illumination sources can be controlled so as to yield a desired combined illumination;

FIG. 13 shows that in certain embodiments, a process can be implemented such that two or more of the illumination sources can be controlled so as to yield a desired sequence of illuminations;

FIG. 14 shows that in certain embodiments, a process can be implemented so as to allow switching between a plurality of viewing modes selected by a user; and

FIG. 15 shows that in certain embodiments, the endoscope system can include a feedback component that facilitates feedback-control of the two or more different color light sources.

These and other aspects, advantages, and novel features of the present teachings will become apparent upon reading the following detailed description and upon reference to the accompanying drawings. In the drawings, similar elements have similar reference numerals.

### DETAILED DESCRIPTION OF CERTAIN EMBODIMENTS

The present disclosure relates generally to medical devices and methods, and in some embodiments, to endoscopes and other devices for viewing and/or imaging objects inside a body. For the purpose of description, a “body” can be that of a human or non-human animal, and can also be that of a living or non-living animal.

Endoscopes are useful tools for viewing and/or imaging objects inside a cavity of a body. Such a cavity can include, for example, a portion of a blood vessel or a gastrointestinal tract. Additional details about endoscopes and components therein can be found in, for example, U.S. patent application Ser. No. 11/099,435 (U.S. Publication No. 2006-0041193) which is incorporated herein by reference in its entirety.

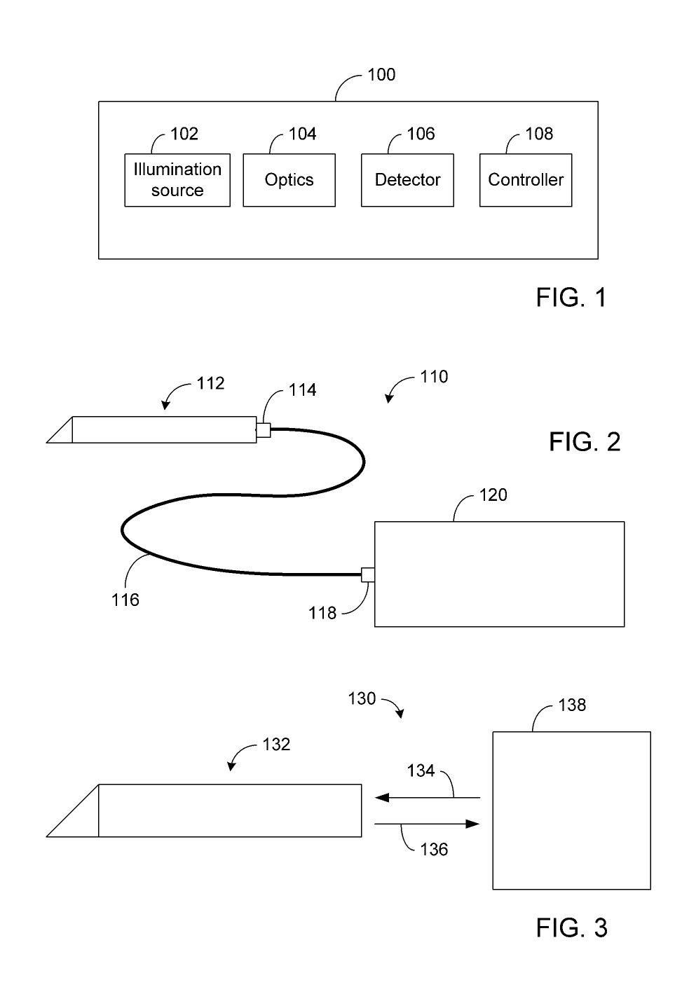

As described herein, the present disclosure provides one or more features that can allow illumination (of an object being observed) from the endoscope to have one or more desirable properties. FIG. 1 shows that in certain embodiments, an endoscope system 100 can include various components that can facilitate generation of illuminations having such desirable properties.

The system 100 can include an illumination source component 102 for providing illumination to a region of interest so as to allow imaging of one or more objects in the region. For the purpose of description, “illumination” is sometimes referred to as “light.” Also for the purpose of description, illumination and/or light can include visible light as commonly understood, as well as wavelength ranges typically associated with ultra-violet and/or infrared radiation. Non-limiting examples of the illumination source component 102 are described herein in greater detail.

For the purpose of description herein, various components are sometimes referred to as “monochromatic” and “single-color.” Also, certain colors are referred to as, for example, “red,” “green,” and “blue.” Typically, an intensity distribution of a given color light can have certain shape and width, and such width can extend to a region typically associated with another color. Thus, terms such as “single-color” can mean predominantly of that color, with the understanding that there may be components associated with other color(s). In the context of the present disclosure, usages of terms such as the foregoing examples are not intended to, and in fact do not, restrict or limit the various concepts described herein.

The system 100 can also include an optics component 104 configured to form images of the illuminated objects. For the purpose of description, it will be understood that such images can result from reflection of light from the object, as well as induced light emission such as fluorescence. Non-limiting examples of the optics component can be found in the herein-mentioned U.S. patent application Ser. No. 11/099,435 which is incorporated herein by reference in its entirety.

The system 100 can also include a detector component 106 configured to detect and capture images formed by the optics component 104. Such a detector can be, for example, a segmented detector such as a charge-coupled-device (CCD) or a complementary-metal-oxide-semiconductor (CMOS) detector. Such a detector can include a detector array with an array of detector elements.

In certain embodiments as described herein, the detector 106 can be configured to operate as a color detector or a monochromatic detector (also sometimes referred to as a black-and-white detector). In certain embodiments, a monochromatic detector can be utilized in conjunction with certain illumination modes of the sources 102 to yield color images. An example of such an operating mode is described herein in greater detail.

The system 100 can also include a controller component 108 configured to provide one or more controlling functionalities of one or more components of the system 100. In certain optional embodiments, the controller component 108 can include a processor, and optionally an associated tangible storage medium, configured to perform or induce performance of such functions.

FIGS. 2 and 3 show that the endoscope system 100 (FIG. 1) can be embodied in a number of ways. For example, FIG. 2 shows that in certain embodiments, a system 110 can include an endoscope probe 112 physically coupled to a separate component 120 via a cable assembly 116.

The probe 112 can include, for example, a light source assembly disposed at its distal end. The probe 112 can also include an optics assembly and a detector to facilitate formation and detection of images of illuminated objects. For such an example endoscope configuration, the cable assembly 116 can include an electrical power supply cable for powering the light source and detector, and a signal cable for transferring signals to and from the same. The electrical power can be supplied by a power source that is either part of, or facilitated by, the separate component 120. The separate component 120 can also include a processor for providing controlling and/or signal processing functionalities. In certain embodiments, the cable assembly 116 can be coupled to one or both of the probe 112 and separate component 120 via connectors (114 and 118) in known manners. In certain embodiments, a detector can also be disposed at proximal end of the component 120 with relay lenses or fiber optic bundle in the cable assembly 116.

In another example, FIG. 3 shows that in certain embodiments, a system 130 can include an endoscope probe 132 configured to communicate with a separate component 138 via a communication link such as a wireless link. In such a system, the probe 132 can be powered by, for example, a battery such that the power connection of FIG. 2 is not needed.

Further, signal transferring functionality can be provided wirelessly. For example, control signals for the light source and/or the detector can be transmitted wirelessly (depicted as arrow 134) from the separate component 138 to the probe 132. Similarly, signals from the detector can be transmitted to the separate component 138 wirelessly (depicted as arrow 136).

A number of other configurations are also possible. For example, some combination of connectivities shown in FIGS. 2 and 3 can be implemented.

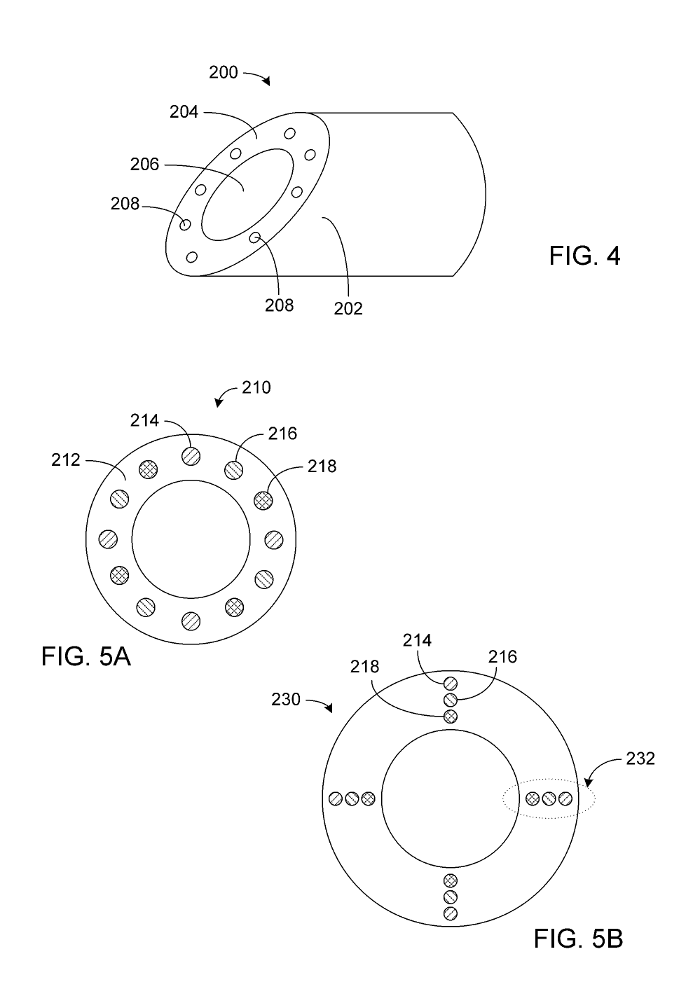

In certain embodiments, the illumination source component 102 of FIG. 1 can be implemented by providing a plurality of different color light sources at a distal end of the probe (e.g., 112 in FIGS. 2 and 132 in FIG. 3). FIG. 4 shows that in certain embodiments, a surface 204 at the distal end 202 of a probe 200 can be angled and provided with light sources 208 such as light-emitting diodes (LEDs) so as to allow side viewing through a viewing window 206. Additional details about such an example configuration can be found in the herein-mentioned U.S. patent application Ser No. 11/099,435 which is incorporated herein by reference in its entirety. Other configurations of the light sources are also possible.

As described herein, the plurality of different color light sources (such as the light sources 208 in FIG. 4) can be controlled to provide one or more desired illumination properties. In certain situations, some of such different modes of illumination can be enhanced or made more efficient by arranging the different color light sources appropriately. Accordingly, FIGS. 5A and 5B show non-limiting examples of how such different color light sources can be arranged.

In FIG. 5A, an example arrangement 210 of different light sources 214, 216, and 218 is depicted as being provided on a distal end surface 212 (of the endoscope probe). For the purpose of description of FIGS. 5A and 5B, three different light sources are depicted. First type of light source 214 (e.g., red LED) is depicted with a slanted hatch pattern; second type 216 (e.g., green LED) with a reverse slanted hatch pattern; and third type 218 (e.g., blue LED) with a cross-hatch pattern. In the example arrangement 210, the three types of sources 214, 216, and 218 are depicted as being positioned circumferentially about the viewing window in repeating sequences (e.g., red, green, blue, red, green, and so on).

In certain embodiments, it may be desirable to position a number of groups of lights about the viewing window, with each group having at least one of each of the different color light sources. Thus, in FIG. 5B, an example arrangement 230 depicts four example groups 232 disposed at four circumferential positions, with each group 232 having one of each of the three types of light sources 214, 216, and 218.

A number of other light source arrangements are also possible.

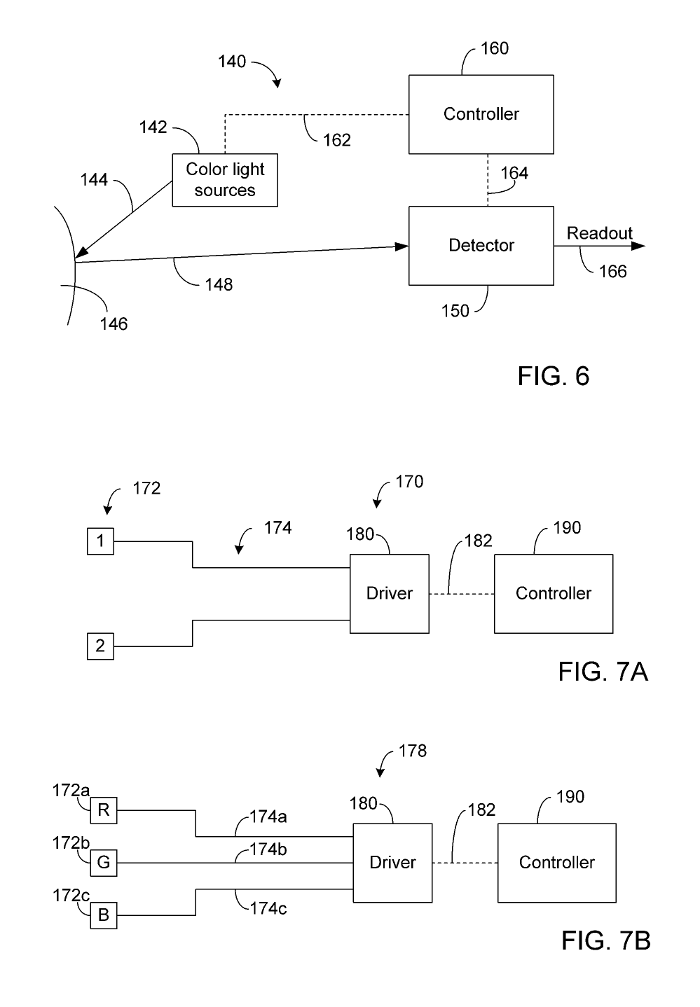

FIG. 6 shows an example situation where an endoscope system 140 is being utilized. An assembly 142 of a plurality of color light sources is depicted as illuminating (arrow 144) an object 146. Reflected light and/or induced light emission (arrow 148) is shown to be detected by a detector 150.

As shown, operation of the light sources 142 and the detector 150 can be controlled (depicted as lines 162 and 164) by a controller 160. The controller 160 can also facilitate reading out of signals (depicted as arrow 166) from the detector 150.

As described herein, controlling of the light sources 142 can be performed so as to yield one or more desired illumination modes. Examples of such illumination modes are described herein in greater detail.

FIG. 7A shows an example of how the light sources can be controlled. In certain embodiments, an illumination configuration 170 can include a driver 180 under control (line 182) of a controller 190. The driver 180 can provide driving signals 174 to different colored light sources (in the example, two sources “1” and “2”) 172.

FIG. 7B shows an example 178 of the illumination configuration of FIG. 7A. In certain embodiments, the driver 180 can be an LED driver that provides driving signals (e.g., 174a, 174b, 174c) to different colored LEDs (e.g., R, G, B) 172a, 172b, 172c.

In certain embodiments, two different colored LEDs can be provided and controlled to yield a desired illumination. In other embodiments, three, four, five, or even more different colored LEDs can be provided and controlled to yield desired illumination configurations. In certain embodiments, a plurality of LEDs can be provided and controlled to yield light having 2, 3, 4, 5, or more color and/or wavelength components (e.g., wavelength peaks) in the output illumination.

In certain embodiments, controlling of the LEDs (such as via the example control configuration of FIG. 7B) can include adjustments of output intensities of one or more of the LEDs. Such adjustments can be utilized to yield a combination of colored lights having a desired intensity profile. Such a desired intensity profile can approximate, for example, a profile associated with a selected light source.

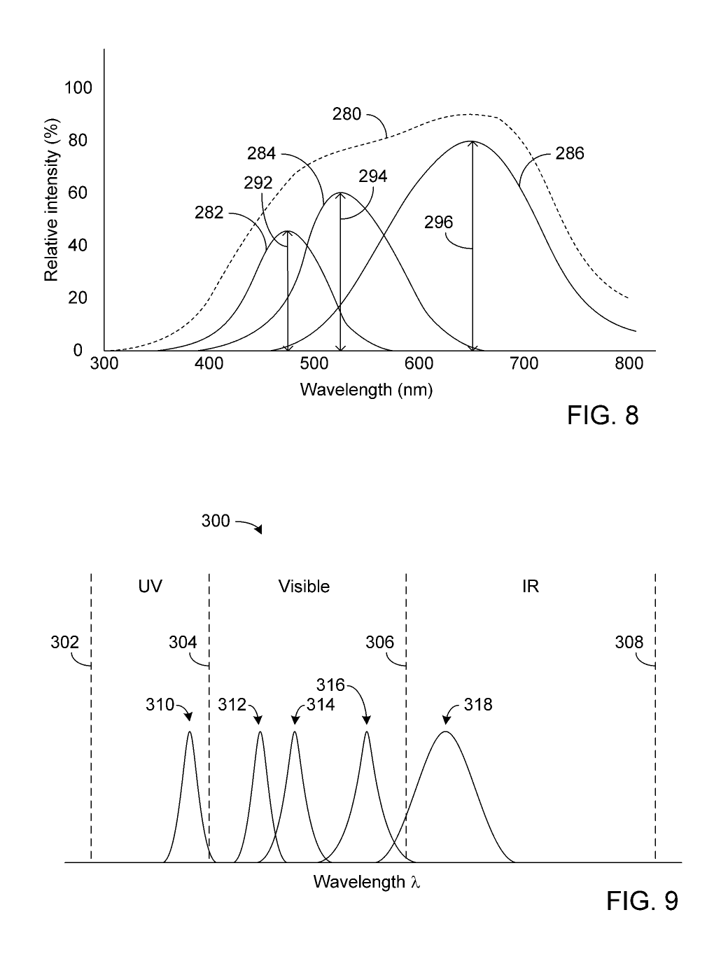

An example of such a selected light source is a Xenon light source that is used in many endoscopic applications, and generally considered to be a standard in color temperature(s) in many of such endoscopic applications. FIG. 8 shows a schematic illustration of a typical Xenon bulb's intensity distribution 280. Also shown are sketches of intensity curves (286, 284, and 282) corresponding to the example red, green, and blue LEDs. The intensity curves 286, 284, and 282 are shown to have intensity amplitudes 296, 294, and 292, respectively. Thus, in certain embodiments, intensity amplitudes of the LEDs can be adjusted (e.g., via the controller and driver of FIG. 7B) so as to yield a desired combined color distribution.

In certain embodiments, the LEDs can be controlled so as to mimic other sources such as, and not limited to, white light sources such as fluorescent bulbs, incandescent bulbs, etc.

In certain embodiments, the illumination sources as described herein can include one or more sources configured to emit at wavelength range(s) associated with ultraviolet and/or infrared radiation. In certain situations, ultraviolet illumination can provide endoscopic viewing modes that may not be achievable via visible light. For example, there may be situations where an object being viewed via the endoscope is detectable through the presence of a fluorescing compound (e.g., fluorescing protein), and such fluorescence may have excitation band(s) in the UV and/or visible range(s). Similarly, there may be situations where infrared or near-infrared illumination can provide a more preferable viewing property than that of visible light.

Thus, FIG. 9 show an example emission spectra 300 that can represent certain embodiments of the illumination sources. As shown, a UV range (typically about 10 nm to 380 nm) is indicated by dashed lines 302 and 304, a visible range (typically about 380 nm to 760 nm) by dashed lines 304 and 306, and an infrared range (typically about 760 nm to 1 mm depending on sub-ranges of IR) by dashed lines 306 and 308. In certain embodiments, the illumination sources can include a plurality of sources having various components from one or more of the UV, visible, and IR ranges.

For example, in certain embodiments, a plurality of sources can all be from the visible range (e.g., three sources 312, 314, and 316). In certain embodiments, a plurality of sources can include one or more from the UV range (e.g., source 310) and one or more from the visible range (e.g., sources 312, 314, and/or 316). In certain embodiments, a plurality of sources can include one or more from the IR range (e.g., source 318) and one or more from the visible range (e.g., sources 312, 314, and/or 316). Various other combinations are possible.

In certain embodiments a plurality of sources can include RGB sources and at least one UV source. In certain embodiments a plurality of sources can include RGB sources and at least one IR source. In certain embodiments a plurality of sources can include RGB sources, at least one UV source, and at least one IR source.

In certain embodiments, a plurality of sources can include a plurality of UV sources that can be controlled so as to yield a desired UV illumination spectrum. Similarly, a plurality of sources can include a plurality of IR sources that can be controlled so as to yield a desired IR illumination spectrum.

In certain situations, use of illumination sources from different spectral ranges can provide flexibility in how certain objects can be examined. For example, suppose that an object being observed responds to fluorescence excitation by UV light. Such viewing mode can be achieved by one or more sources in the UV range. It may also be desirable to view the same object under visible light substantially without the fluorescence effect. For such a viewing mode, the UV source(s) can be switched off, and one or more sources in the visible range can be activated. An example of how such selective viewing modes can be controlled is described herein in greater detail.

In certain situations, it may be desirable to view an object with different modes of visible light. Thus, in certain embodiments, two or more different color light sources can be provided and controlled so as to yield illumination having a desired color temperature.

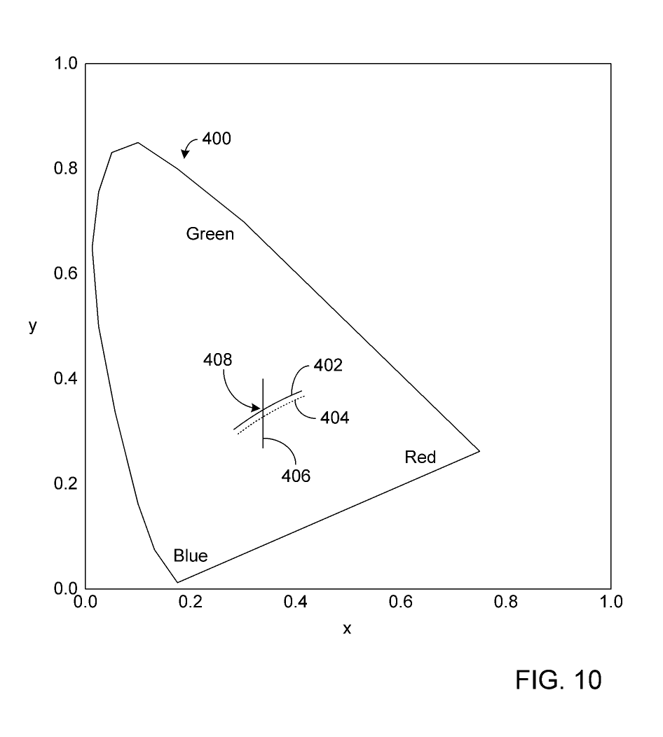

As an example, FIG. 10 shows a schematic illustration of a color space 400 associated with visible light. The “x” and “y” coordinates represent chromaticity coordinates as defined by the International Commission on Illumination (usually abbreviated CIE for its French name). Within the color space 400, a black body chromaticity curve 404 is depicted as a dotted line, and a chromaticity curve 408 representative of standardized D-series illuminants is depicted as a solid line 402. Intersecting the D-illuminant curve 408 is an example isothermal color temperature line 406. As an example, an intersection 408 where the D-illuminant curve 408 and an isothermal color temperature curve for 6504 K (represented as correlated color temperature or “CCT”) is referred to as a “D65” white point. Other notable D-series white points include D55 (CCT of 5503 K) and D75 (CCT of 7504). These three example white points D55, D65, and D75 are typically associated with natural daylight.

In certain embodiments, two or more different color light sources can be provided and controlled so as to yield or approximate one or more standardized white points such as the D-series whitepoints. Other standardized white points are also possible. Table 1 lists some non-limiting example white-points.

TABLE 1

| Name | x | y | CCT (K) | Type of visible light |
|---|---|---|---|---|
| A | 0.44757 | 0.40745 | 2856 | Incandescent/Tungsten |
| B | 0.34842 | 0.35161 | 4874 | Direct sunlight at noon |
| C | 0.31006 | 0.31616 | 6774 | Average/North sky Daylight |
| D50 | 0.34567 | 0.35850 | 5003 | Horizon Light. ICC profile PCS |
| D55 | 0.33242 | 0.34743 | 5503 | Mid-morning/Mid-afternoon |
| | | | | Daylight |
| D65 | 0.31271 | 0.32902 | 6504 | Noon Daylight: Television, |
| | | | | sRGB color space |
| D75 | 0.29902 | 0.31485 | 7504 | North sky Daylight |
| E | 1/3 | 1/3 | 5454 | Equal energy |
| F1 | 0.31310 | 0.33727 | 6430 | Daylight Fluorescent |
| F2 | 0.37208 | 0.37529 | 4230 | Cool White Fluorescent |
| F3 | 0.40910 | 0.39430 | 3450 | White Fluorescent |
| F4 | 0.44018 | 0.40329 | 2940 | Warm White Fluorescent |
| F5 | 0.31379 | 0.34531 | 6350 | Daylight Fluorescent |
| F6 | 0.37790 | 0.38835 | 4150 | Lite White Fluorescent |
| F7 | 0.31292 | 0.32933 | 6500 | D65 simulator, Daylight simulator |
| F8 | 0.34588 | 0.35875 | 5000 | D50 simulator, Sylvania F40 |
| | | | | Design 50 |
| F9 | 0.37417 | 0.37281 | 4150 | Cool White Deluxe Fluorescent |
| F10 | 0.34609 | 0.35986 | 5000 | Philips TL85, Ultralume 50 |
| F11 | 0.38052 | 0.37713 | 4000 | Philips TL84, Ultralume 40 |
| F12 | 0.43695 | 0.40441 | 3000 | Philips TL83, Ultralume 30 |

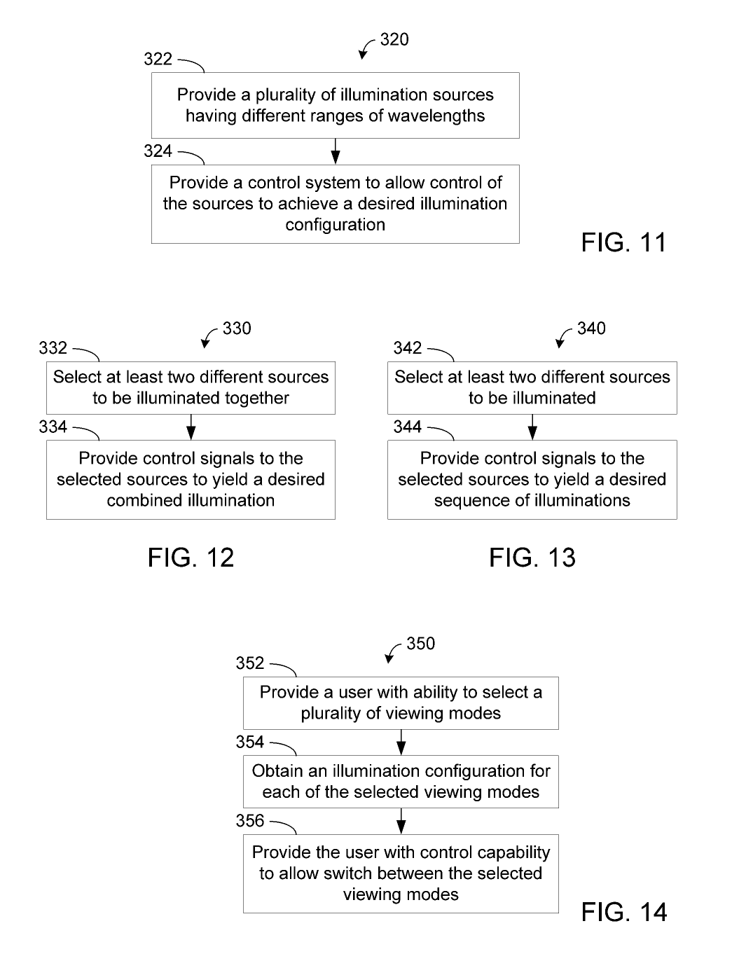

FIG. 11 shows that in certain embodiments, a process 320 can be implemented to facilitate controlling of illumination sources to achieve various viewing modes. In a process block 322, a plurality of illumination sources having different ranges of emission wavelengths can be provided. In a process block 324, a control system (e.g., control circuitry) can be provided to facilitate controlling of the illumination sources so as to achieve a desired illumination configuration suited for one or more viewing modes.

There are a number of illumination configurations that a user may want. FIGS. 12-14 show processes that can be implemented to achieve some non-limiting examples of such illumination configurations.

In certain situations, it may be desirable to combine outputs of two or more sources so as to yield a desired combination. Thus, a process 330 of FIG. 12 can be implemented to achieve such an illumination configuration. In a process block 332, at least two different sources can be selected, where the selected sources are to provide illumination together. In a process block 334, control signals can be provided to the selected sources to yield desired outputs from the sources, such that illuminations from the sources combine to yield a desired illumination. FIG. 8 depicts an example of an illumination configuration that can be achieved by the process 330.

In certain situations, it may be desirable to provide a sequence of illuminations using two or more different sources. Thus, a process 340 of FIG. 13 can be implemented to achieve such an illumination configuration. In a process block 342, at least two different sources can be selected. In a process block 344, control signals can be provided to the selected sources to yield desired outputs from the sources, such that illuminations from the sources are in some desired sequence. In certain situations, an object can be sequentially illuminated with different color light sources, and resulting monochromatic images can be detected by a monochromatic detector. In certain implementations, such images can be combined by a controller so as to yield a color image. As is generally known, some monochromatic detectors can provide higher resolution capability than similarly priced color detectors. Thus, such a technique can provide a relatively high resolution color image by combining two or more monochromatic images generated by the monochromatic detector.

In certain situations, it may be desirable to provide an endoscope system with a plurality of viewing modes. Processes described in reference to FIGS. 12 and 13 can be considered to be examples of such viewing modes. Further, operation of the endoscope in different wavelength ranges (e.g., UV, visible, IR) as described in reference to FIG. 9 can also be considered to be different viewing modes. Also, operation of the endoscope in visible-light illumination configurations (as described in reference to FIGS. 8-10) can be considered to be different viewing modes. For example, one may wish to view an object with Xenon-like illumination (first viewing mode) and with a white point (such as one of the standardized white points (e.g., D55, D65, and D75)) (second viewing mode). In another example, one may wish to switch between different standardized white points.

Thus, a process 350 of FIG. 14 can be implemented to provide a user with a capability to endoscopically view an object in a plurality of modes, and to switch between such modes. In a process block 352, a user can be provided with an ability to select a plurality of viewing modes. In a process block 354, illumination configurations corresponding to the selected viewing modes can be obtained. In a process block 356, the user can be provided with control capability to switch between the selected viewing modes. In certain embodiments, such control capability can be facilitated by an input device such as a switch, computer, keyboard, keypads, touch-screen, etc.

In certain embodiments, control of the different color light sources can be facilitated by a feedback system. For example, a given illumination mode being used may, for whatever reason, result in the observed light being different from a desired profile. Such a difference can be detected, and the light source control can be adjusted to compensate for the difference.

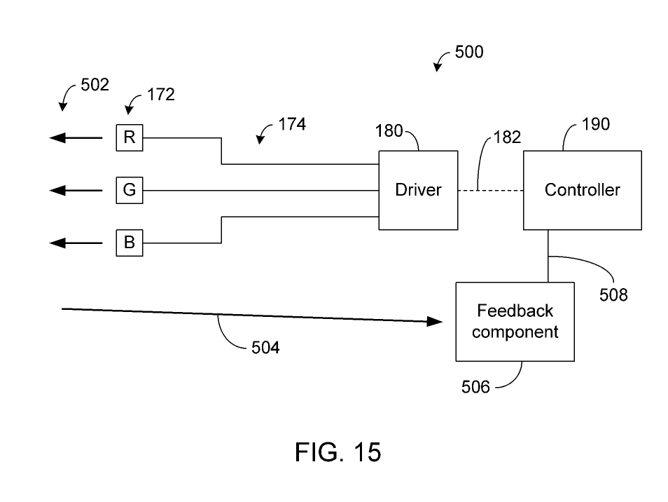

FIG. 15 shows an example of how such feedback control can be achieved. As already described herein in reference to FIG. 7B, a plurality of light sources such as RGB LEDs 172 can be controlled by a controller 190 via a driver 180. Such control of the LEDs 172 is shown to result in color light emissions 502 that combine to yield in a selected viewing mode.

As also shown, light (arrow 504) from an object (not shown) being observed is depicted as being detected by a feedback component 506. Such light 504 can be reflected light and/or emitted light (e.g., fluorescence) from the object in response to the light 502 from the sources 172. The feedback component can be configured to analyze the detected light to determine whether the controlled light output 502 is acceptable. Such analysis can be based on, for example, intensities of various wavelength components in the detected light 504.

In certain embodiments, the detection of the light 504 can be achieved by the same detector used for endoscopic viewing purpose (e.g., detector 150 in FIG. 6). In certain embodiments, the detection of the light 504 can be achieved by a detector that is separate from that used for endoscopic viewing purpose.

As shown in FIG. 15, the feedback component 506 can be in communication (line 508) with a controller 190 so as to induce an adjustment if the detected light is not acceptable. For example, suppose that in a Xenon-like viewing mode, the red component (e.g., 286 in FIG. 8) has an intensity below a threshold level. Then, the feedback component 506 can inform the controller 190 of the analysis, and the controller 190 can make appropriate adjustments such that the detected light 504 has desired properties.

In one or more example embodiments, the functions, methods, algorithms, techniques, and components described herein may be implemented in hardware, software, firmware (e.g., including code segments), or any combination thereof. If implemented in software, the functions may be stored on or transmitted over as one or more instructions or code on a computer-readable medium. Tables, data structures, formulas, and so forth may be stored on a computer-readable medium. Computer-readable media can be non-transitory, and can include both computer storage media and communication media including any medium that facilitates transfer of a computer program from one place to another. A storage medium may be any available medium that can be accessed by a general purpose or special purpose computer. By way of example, and not limitation, such computer-readable media can comprise RAM, ROM, EEPROM, CD-ROM or other optical disk storage, magnetic disk storage or other magnetic storage devices, or any other medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, includes compact disc (CD), laser disc, optical disc, digital versatile disc (DVD), floppy disk and bluray disc where disks usually reproduce data magnetically, while discs reproduce data optically with lasers. Combinations of the above should also be included within the scope of computer-readable media.

For a hardware implementation, one or more processing units at a transmitter and/or a receiver may be implemented within one or more computing devices including, but not limited to, application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

For a software implementation, the techniques described herein may be implemented with code segments (e.g., modules) that perform the functions described herein. The software codes may be stored in memory units and executed by processors. The memory unit may be implemented within the processor or external to the processor, in which case it can be communicatively coupled to the processor via various means as is known in the art. A code segment may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

Although the above-disclosed embodiments have shown, described, and pointed out the fundamental novel features of the invention as applied to the above-disclosed embodiments, it should be understood that various omissions, substitutions, and changes in the form of the detail of the devices, systems, and/or methods shown may be made by those skilled in the art without departing from the scope of the invention. Consequently, the scope of the invention should not be limited to the foregoing description, but should be defined by the appended claims.

All publications and patent applications mentioned in this specification are indicative of the level of skill of those skilled in the art to which this invention pertains. All publications and patent applications are herein incorporated by reference to the same extent as if each individual publication or patent application was specifically and individually indicated to be incorporated by reference.

What is claimed is:

1. An endoscope system, comprising:
a probe configured to be insertable into a body;
a plurality of different illumination sources configured and disposed relative to said probe so as to provide illumination having one or more desired properties from said probe to an object inside said body;
an assembly of optical elements configured and disposed relative to said probe so as to form images of said illuminated object; and
a controller configured to control operation of said illumination sources so as to yield said one or more desired properties of said illumination.

2. The system of claim 1, further comprising a detector configured to detect said images and generate signals representative of said detected images.

3. The system of claim 1, wherein said illumination sources comprise different color light sources.

4. The system of claim 3, wherein said different color light sources are configured to yield a desired white light.

5. The system of claim 3, wherein said different color light sources comprise red (R), green (G), and blue (B) color light sources.

6. The system of claim 5, wherein output intensity of each of said R, G, and B color light sources is controlled independently so as to provide a desired white light.

7. The system of claim 6, wherein said white light comprises chromacity values associated with one or more standardized white points.

8. The system of claim 7, wherein said one or more standardized white points comprise one or more of D55, D65, and D75 white points.

9. The system of claim 1, wherein said illumination sources comprise light-emitting diodes (LEDs).

10. The system of claim 9, wherein at least two of said LEDs are configured to emit light substantially in a wavelength range corresponding to visible light.

11. The system of claim 9, wherein at least one of said LEDs is configured to emit substantially in a wavelength range corresponding to visible light, and at least one of said LEDs is configured to emit substantially in a wavelength range corresponding to ultraviolet radiation.

12. The system of claim 9, wherein at least one of said LEDs is configured to emit substantially in a wavelength range corresponding to visible light, and at least one of said LEDs is configured to emit substantially in a wavelength range corresponding to infrared radiation.

13. The system of claim 1, wherein said illumination sources are disposed on said probe.

14. The system of claim 13, wherein said illumination sources are disposed at or near a distal end of said probe.

15. The system of claim 1, wherein said controller is configured so as to allow control operation of each of said illumination sources separately.

16. The system of claim 1, wherein said controller is configured to provide control signals to two or more of said different illumination sources, each of said control signals resulting in a corresponding illumination source to operate in a selected manner such that combination of outputs from said two or more sources has a desired illumination property.

17. The system of claim 10, wherein said desired illumination property comprises an approximation of an intensity distribution associated with a Xenon light source.

18. The system of claim 10, wherein said desired illumination property comprises an approximation of an intensity distribution associated with a fluorescent light source.

19. The system of claim 10, wherein said desired illumination property comprises an approximation of an intensity distribution associated with an incandescent light source.

20. The system of claim 1, wherein said controller is configured to provide control signals to two or more of said different illumination sources so as to yield sequential activation of said two or more sources.

21. The system of claim 20, wherein said two or more sources comprise two or more substantially single-color light sources such that said object is illuminated by a sequence of said substantially single-color lights.

22. The system of claim 21, wherein said detector comprises a substantially monochromatic detector such that detection of substantially monochromatic images resulting from said single-color illuminations allows combination of said monochromatic images to yield a color image.

23. The system of claim 1, wherein said controller is configured to provide control signals to two or more of said different illumination sources in a manner that allows switching between two or more viewing modes facilitated by said two or more illumination sources.

24. A method for configuring an endoscope, comprising:
providing a plurality of different illumination sources; and
providing a control mechanism configured to control each of said illumination sources to achieve a desired illumination configuration.

25. The method of claim 24, wherein said control of each source comprises providing said source with an operating signal such that combination of outputs from said sources yields a desired combined illumination.

26. The method of claim 24, wherein said control of each source comprises providing said source with an operating signal that results in said sources being activated in a selected sequence.

27. The method of claim 24, wherein said control mechanism comprises one or more features that allow switching from two or more illumination modes associated with said sources.

28. A method for operating an endoscope, comprising:
providing a plurality of different color light sources;
activating said different color light sources in a manner that yields a selected configuration of illumination provided to an object being examined; and
obtaining an image of said object during at least a portion of said illumination.
